(19) United States
(12) Patent Application Publication
(10) Pub. No.: US 2019/0178862 A1
(43) Pub. Date: Jun. 13, 2019

(54) AIR QUALITY MEASURING APPARATUS

(71) Applicant: POINT ENGINEERING CO., LTD., Asan (KR)

(72) Inventors: Bum Mo AHN, Suwon (KR); Seung Ho PARK, Hwaseong (KR); Sung Hyun BYUN, Hwaseong (KR)

(73) Assignee: POINT ENGINEERING CO., LTD., Asan (KR)

(21) Appl. No.: 16/201,891

(22) Filed: Nov. 27, 2018

(30) Foreign Application Priority Data
Dec. 13, 2017 (KR) 10-2017-0171576

(51) Int. Cl. G01N 33/00; G01N 21/88

(52) U.S. Cl. CPC G01N 33/0016; G01N 21/88; G01N 33/0031

(57) ABSTRACT

The present invention relates to an air quality measuring apparatus. The air quality measuring apparatus measures air quality using air flow generated therein using a variable and restorable pumping means, thereby reducing power consumption and a volume of the air quality measuring apparatus so as to be portable.

### CROSS REFERENCE TO RELATED APPLICATION

The present application claims priority to Korean Patent Application No. 10-2017-0171576, filed Dec. 13, 2017, the entire contents of which is incorporated herein for all purposes by this reference.

### BACKGROUND OF THE INVENTION

### Field of the Invention

The present invention generally relates to an air quality measuring apparatus and, more particularly, to an air quality measuring apparatus that is portable and measures air quality by introducing and discharging surrounding air of the air quality measuring apparatus.

### Description of the Related Art

Generally, fine dust is particulate matter (PM) floating in the atmosphere or blowing down to the ground, and the fine dust primarily occurs from burning of fossil fuels such as coal and oil or from emission gas generated from industrial plants and vehicles.

Since fine dust particulate matter (hereinafter, referred to as “particulate matter”) is invisibly very fine, the particulate matter floats in the air and penetrates lungs through respiratory organs or moves through blood vessels into the deepest parts of the human body, which can cause health problems.

In addition to the particulate matter, sick house syndrome has occurred according to increase of new buildings due to the new town development, and interest in volatile organic compound (VOC), which causes the sick house syndrome, also has increased.

For such a reason described above, with increasing interest in air quality, an air quality measuring apparatus has been actively developed.

As a related-art document of the present invention, Korean Patent No. 10-1765663 (registered Aug. 1, 2017, and referred to as “patent document 1” hereinbelow) is known.

The air quality measuring apparatus of patent document 1 introduces outside air into the air quality measuring apparatus through a blower fan provided in a main body, and measures air quality of the outside air.

In the case of the air quality measuring apparatus of patent document 1, since the blower fan is installed inside the main body of the apparatus, the air quality measuring apparatus generates noise and uses excessive amount of power to operate the fan.

Also, since the volume of the air quality measuring apparatus increases due to the installation of the fan in the main body, the air quality measuring apparatus of patent document 1 has problems such as inconvenience in movement of the apparatus and reduced space efficiency.

Accordingly, the structure using the blower fan is not efficiently used as a portable air quality measuring apparatus.

The foregoing is intended merely to aid in the understanding of the background of the present invention, and is not intended to mean that the present invention falls within the purview of the related art that is already known to those skilled in the art.

### DOCUMENTS OF RELATED ART

(Patent Document 1) Korean Patent No. 10-1765663 (Aug. 1, 2017)

### SUMMARY OF THE INVENTION

Accordingly, the present invention has been made keeping in mind the above problems occurring in the related art, and the present invention is intended to propose an air quality measuring apparatus, which generates air flow using a variable and restorable pumping means and measures air quality, thereby reducing power consumption and reducing the volume of the air quality measuring apparatus to provide a portable apparatus.

In order to achieve the above object, according to an aspect of the present invention, there is provided an air quality measuring apparatus including: an air flow passage communicating with an air inlet and an air outlet; a pumping part disposed on the air flow passage, and configured to introduce outside air into the air flow passage through the air inlet and to discharge the outside air through the air outlet; and a sensor disposed on the air flow passage, and measuring particles in the outside air, when the pumping part is operated to be pressurized, air inside the pumping part is discharged through the air outlet, and then outside air is introduced into the air flow passage through the air inlet while the pumping part is restored to an original shape thereof.

Here, the pumping part may be made of an elastic material.

In addition, the air flow passage may include a first chamber communicating with the air inlet, a second chamber communicated with the air outlet, and a pumping chamber provided in the pumping part, and the pumping chamber may be disposed between the first chamber and the second chamber.

In addition, a first check valve may be provided between the first chamber and the pumping chamber, and a second check valve may be provided between the second chamber and the pumping chamber.

In addition, the sensor may include a gas sensor and a particulate matter sensor, and the gas sensor and the particulate matter sensor may be operated when the outside air is introduced into the air flow passage through the air inlet.

As described above, the air quality measuring apparatus according to the embodiment of the present invention has following effects.

Unlike the air quality measuring apparatus of the related art that generates an air flow using a fan, the air quality measuring apparatus of the present invention can simplify the configuration of the apparatus and reduce power consumption to operate the fan.

In addition, with the simplification of the configuration of the apparatus, the air quality measuring apparatus of the present invention can be made to be used as a portable apparatus by reducing the volume thereof and by being installed in mobile electronic devices such as mobile phone cases.

### BRIEF DESCRIPTION OF THE DRAWINGS

FIG. 1A is a sectional view showing installation of an air quality measuring apparatus in a smart phone case according to an exemplary embodiment of the present invention.

FIG. 1B is a sectional view showing the air quality measuring apparatus according to the exemplary embodiment of the present invention.

FIG. 2 is a sectional view showing a state in which external force is applied to a pumping part of the air quality measuring apparatus of FIGS. 1A and 1B, and a second chamber is opened.

FIG. 3 is a sectional view showing a state of a first chamber opened by suction force of the pumping part of the air quality measuring apparatus of FIGS. 1A and 1B.

### DETAILED DESCRIPTION OF THE INVENTION

Hereinbelow, exemplary embodiments of the present invention will be described in detail with reference to the accompanying drawings.

In the following description, only the theory of the present invention will be described. Thus, although some embodiments are not clearly described in the following description or not clearly shown in the accompanying drawings, those skilled in the art can provide various apparatuses that may be configured to embody the theory of the present invention within the scope and spirit thereof. Further, the terminology used herein is for the purpose of describing particular aspects or embodiments of the present invention only and is not intended to be limiting of the present invention.

The above and other objects, features and other advantages of the present invention will be more clearly understood from the following detailed description when taken in conjunction with the accompanying drawings, and those skilled in the art will easily understand and may embody the scope and spirit of the invention.

An air quality measuring apparatus of the present invention is a portable air quality measuring apparatus, and is able to be installed inside any portable electronic devices in addition to mobile phone cases.

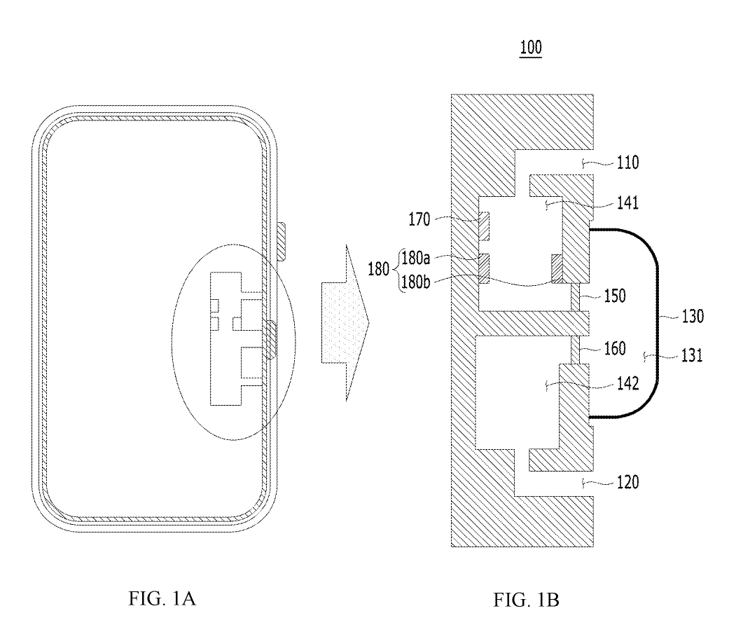

As shown in FIG. 1A, the air quality measuring apparatus of the present invention, which will be described below, is related to an apparatus installed in the mobile phone cases, but application of the present invention is not limited by the mobile phone cases.

Particles included in air, as described below, include gas particles, particulate matters, and moisture particles, and stay in the air as solid, liquid, and gaseous states.

First, an air quality measuring apparatus 100 according to an exemplary embodiment of the present invention will be described with reference to FIGS. 1A, 1B, 2, and 3.

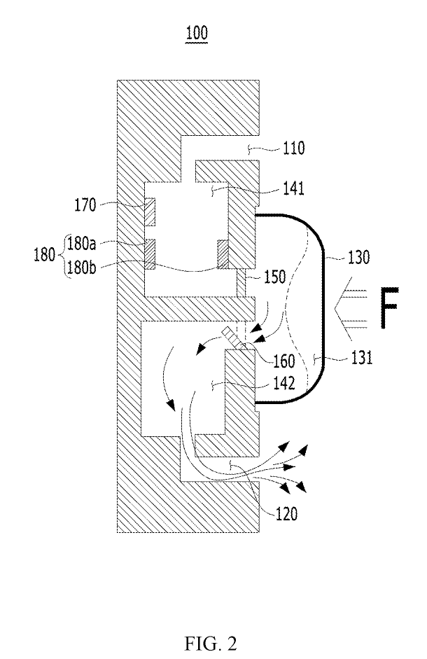

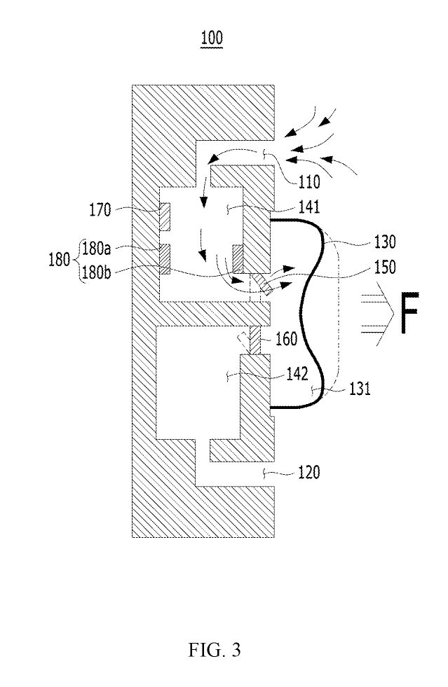

FIG. 1A is a sectional view showing installation of the air quality measuring apparatus in a smart phone case according to the exemplary embodiment of the present invention, and FIG. 1B is a sectional view of the air quality measuring apparatus according to the exemplary embodiment of the present invention, FIG. 2 is a sectional view showing a state in which external force is applied to a pumping part of the air quality measuring apparatus of FIGS. 1A and 1B, and a second chamber is opened, and FIG. 3 is a sectional view showing a state of a first chamber opened by suction force of the pumping part of the air quality measuring apparatus of FIGS. 1A and 1B.

As shown in FIGS. 1A to 3, according to the exemplary embodiment of the present invention, the air quality measuring apparatus 100 includes an air inlet 110 and an air outlet 120 provided in the air quality measuring apparatus 100, an air flow passage provided between the air inlet 110 and the air outlet 120, a sensor disposed on the air flow passage and measuring particles in outside air, and a pumping part 130 configured to discharge inside air of the pumping part 130 through the air outlet 120 when the pumping part is operated to be pressurized, and to newly introduce outside air into the air flow passage through the air inlet 110 when the pumping part is restored to an original shape thereof.

As shown in FIG. 1B, the air flow passage functions as a passage communicating the air inlet 110 and the air outlet 120 with each other, and the air flow passage includes a first chamber 141 communicating with the air inlet 110, a second chamber 142 communicating with the air outlet 120, and a pumping chamber 131 provided in the pumping part 130. Here, the pumping chamber 131 is disposed between the first chamber 141 and the second chamber 142.

Thus, the above-mentioned components are disposed in the air flow passage, from a first end of the air flow passage to a second end thereof, in the order of the air inlet 110, the first chamber 141, the pumping chamber 131 of the pumping part 130, the second chamber 142, and the air outlet 120.

The air inlet 110 communicates with the first chamber 141, and functions as a passage for introducing outside air into the first chamber 141 when the pumping part 130 is restored to the original shape thereof after having been pressurized.

The air outlet 120 communicates with the second chamber 142, and functions as a passage for discharging inside air of the pumping chamber 131 to the outside when the pumping part 130 is pressurized.

The first chamber 141, of which a first end communicates with the air inlet 110, and a second end communicates with the pumping chamber 131, functions to connect the air inlet 110 to the pumping chamber 131.

Because of this first chamber 141, when the pumping part 130 is restored to the original shape thereof after having been operated to be pressurized, the outside air introduced through the air inlet 110 may easily flow into the air quality measuring apparatus 100, that is, the inside of the pumping chamber 131 of the pumping part 130.

A sensor that measures particles in the outside air is provided in the first chamber 141, the sensor will be described in detail later herein.

The second chamber 142 is configured such that a first end thereof communicates with the pumping chamber 131 and a second end thereof communicates with the air outlet 120, thereby connecting the pumping chamber 131 and the air outlet 120 to each other.

Because of this second chamber 142, when the pumping part 130 is pressurized, the inside air staying in the air quality measuring apparatus 100, that is, the inside air staying in the pumping chamber 131, is easily discharged to the outside through the air outlet 120.

The air quality measuring apparatus 100 has check valves to make air flow in one direction.

The check valves are operated by the air flow due to compression force and the suction force, and are normally closed to separate the first chamber 150, the pumping chamber 131, and the second chamber 160 from each other.

The check valves may include a first check valve 150 and a second check valve 160, wherein each of the first and second check valves 150 and 160 is configured to be opened in one direction.

The first check valve 150 is provided between the first chamber 141 and the pumping chamber 131, and is normally closed to separate the first chamber 141 and the pumping chamber 131 from each other.

When the pumping part 130 is restored to the original shape thereof after having been operated to be pressurized, the first check valve 150 is opened only in a direction toward the pumping chamber 131 by the suction force generated in the air quality measuring apparatus 100. When the pumping part 130 is operated to be pressurized, the first check valve 150 prevents the inside air staying in the pumping chamber 131 from flowing to the first chamber 141.

The second check valve 160 is provided between the pumping chamber 131 and the second chamber 142, and is normally closed to separate the pumping chamber 131 and the second chamber 142 from each other.

The second check valve 160 is opened only in a direction toward the second chamber 142 when the pumping part 130 is operated to be pressurized, and is closed when the pumping part 130 is restored to the original shape thereof after having been operated to be pressurized, thereby preventing that the air flow moves from the second chamber 141 to the pumping chamber 131.

As described above, by the check valves that are provided between the pumping chamber 131 and the first chamber 141 and between the pumping chamber 131 and the second chamber 142 and are opened in one direction, the first chamber 141 and the second chamber 142 does not communicate with each other. Therefore, the air flow in the air quality measuring apparatus 100 can be induced in one direction.

The pumping part 130 is a device for making the air flow in the air quality measuring apparatus 100.

The pumping part 130 may be made of elastic materials, for example, rubber, but the present invention is not limited to the materials.

The pumping part 130 is provided on a side surface of the air quality measuring apparatus 100, and is formed within a range including the first check valve 150 and the second check valve 160.

The pumping chamber 131 is an inside space, made of elastic materials, of the pumping part 130, and is disposed between the first chamber 141 and the second chamber 142.

The pumping chamber 131 communicates with the first chamber 141 and the second chamber 142, the check valves are respectively provided between the pumping chamber 131 and the first chamber 141 and between the pumping chamber 131 and the second chamber 142.

The pumping chamber 131 prevents that the outside air, that is the air to be measured for air quality, and the inside air staying in the air quality measuring apparatus 100 are mixed with each other.

Described in detail, the outside air, introduced into the air quality measuring apparatus 100 through the air inlet 110 due to the suction force of the pumping part 130, passes through the first chamber 141 and stays in the pumping chamber 131, and then passes through the second chamber 142 due to the compression force, and is discharged to the outside through the air outlet 120.

That is, when the pumping part 130 is operated to be pressurized, the inside air staying in the pumping part 130 is discharged through the air outlet 120, and then new outside air is introduced into the air quality measuring apparatus 100 through the air inlet 110 while the pumping part 130 is restored to the original shape thereof.

Accordingly, due to the pumping chamber 131, the air quality measuring apparatus 100 can provide accurate results of measurement of air quality.

The pumping part 130 described above is provided on the side of the air quality measuring apparatus 100, as shown in FIG. 1B.

Hereinbelow, the sensors provided in the first chamber 141 will be described.

The sensors, which measure the particles inside air, may include a gas sensor 170 and a particulate matter sensor 180, as an example.

As shown in FIG. 1B, the gas sensor 170 is made of one sensor, and is disposed on a side surface inside the first chamber 141.

The gas sensor 170 may measure pollutants in the air using a sensing film that is configured to react with specific substances. In addition, since the moisture particles in the air is removed by a heater included in the gas sensor, an error that may be caused by mistakenly measuring the moisture particles as the particulate matters can be prevented in advance.

The particulate matter sensor 180 is made of a light emitter 180a and a light receiver 180b, as shown in FIG. 1B, wherein the light emitter 180a and the light receiver 180 are disposed on opposite side surfaces in the first chamber 141.

The light emitter 180a and the light receiver 180 are operated by a method, in which when the light emitter 180a provided on one side surface emits light, the light receiver 180b provided on a remaining side surface detects the light.

Described in detail, when introduced air passes through the particulate matter sensor 180, light emitted from the light emitter 180a is scattered by the particulate matters in the introduced air.

As the light is scattered by the particulate matters, amount of light reaching the light receiver 180b varies, therefore concentration of the particulate matters is measured by detecting the amount of the light reaching the light receiver 180b.

That is, when the concentration of the particulate matters in the air is high, the light reaching the light receiver 180b is detected in a small amount, and when the concentration of the particulate matters in the air is low, the light reaching the light receiver 180b is detected in a large amount.

In a measurement method of the concentration of the particulate matters as described above, error may occur because the light is also scattered by the moisture particles in the air.

Consequently, as the moisture particles in the air are removed by the heater provided in the gas sensor, the error can be greatly reduced.

Therefore, to accurately measure particulate matters in the air, the sensors provided in the first chamber 141 are installed in the order from the gas sensor 170 to the particulate matter sensor 180.

Hereinbelow, referring to FIGS. 2 and 3, a method of measuring air quality in outside air by using the air quality measuring apparatus 100 having an above-described configuration will be described.

When outside force F is not applied to the air quality measuring apparatus 100, the first check valve 150 and the second check valve 160 are maintained in closed states.

As shown in FIG. 2, when the pumping part 130 is operated to be pressurized, air staying in the pumping chamber 131 passes through the second chamber 142, and is discharged to the outside through the air outlet 120.

That is, by the compression force applied to the pumping part 130, the second check valve 160 is opened in a direction toward the inside of the second chamber 142, and then the air staying in the pumping chamber 131 is introduced into the second chamber 142. In this case, the first check valve 150 is not opened.

As shown in FIG. 3, when the outside force F applied to the pumping part 130 is released, the pumping part 130 is restored to an original shape thereof.

As the pumping part 130 is restored to the original shape thereof, the suction force is generated in the air quality measuring apparatus 100.

That is, outside air is introduced into the air quality measuring apparatus 100 through the air inlet 110 by the suction force. In this case, the second check valve 160 is closed by the suction force and the first check valve 150 is opened in the direction toward the pumping chamber 131.

Here, the particles in the outside air, introduced through the air inlet 110, are measured using the gas sensor 170 and the particulate matter sensor 180 provided in the first chamber 141.

The outside air after finishing the measurement stays in the pumping chamber 131, and is discharged to the outside by the compression force of the pumping part 130 operated to be pressurized later to measure particulate matters in new air.

That is, since the compression force and the suction force of the pumping part 130 are used to discharge the air staying in the air quality measuring apparatus 100 through the air outlet 120 and to introduce outside air for measuring air quality, the present invention can more easily perform the air quality measurement of the outside air.

As described above, according to the exemplary embodiment of the present invention, since the unidirectional air flow is generated in the air quality measuring apparatus 100 by the compression force and the suction force, which are generated due to elasticity of the pumping part 130 made of elastic materials, the air quality measuring apparatus 100 of the present invention can simplify the configuration thereof and reduce power consumption, unlike the conventional air quality measuring apparatus that is configured to generate air flow using a fan.

In addition, as the configuration of the air quality measuring apparatus 100 is simplified, the volume of the apparatus 100 can be reduced, and manufactured as a portable apparatus capable of being installed in mobile electronic devices such as mobile phone cases.

Although a preferred embodiment of the present invention has been described for illustrative purposes, those skilled in the art will appreciate that various modifications, additions and substitutions are possible, without departing from the scope and spirit of the invention as disclosed in the accompanying claims.

What is claimed is:

1. An air quality measuring apparatus comprising:
an air flow passage communicating with an air inlet and an air outlet;
a pumping part disposed on the air flow passage, and configured to introduce outside air into the air flow passage through the air inlet and to discharge the outside air through the air outlet; and
a sensor disposed on the air flow passage, and measuring particles in the outside air,
wherein, when the pumping part is operated to be pressurized, air inside the pumping part is discharged through the air outlet, and then outside air is introduced into the air flow passage through the air inlet while the pumping part is restored to an original shape thereof.

2. The air quality measuring apparatus of claim 1, wherein the pumping part is made of an elastic material.

3. The air quality measuring apparatus of claim 1, wherein the air flow passage comprises a first chamber communicating with the air inlet, a second chamber communicated with the air outlet, and a pumping chamber provided in the pumping part, and the pumping chamber is disposed between the first chamber and the second chamber.

4. The air quality measuring apparatus of claim 3, wherein a first check valve is provided between the first chamber and the pumping chamber, and a second check valve is provided between the second chamber and the pumping chamber.

5. The air quality measuring apparatus of claim 1, wherein the sensor comprises a gas sensor and a particulate matter sensor, and the gas sensor and the particulate matter sensor are operated when the outside air is introduced into the air flow passage through the air inlet.
